(19) United States
(12) Patent Application Publication
(10) Pub. No.: US 2021/0275364 A1
(43) Pub. Date: Sep. 9, 2021

(54) ELASTOMERIC LAMINATE WITH CONTROL LAYER AND METHODS THEREOF

(71) Applicant: The Procter & Gamble Company, Cincinnati, OH (US)

(72) Inventors: Todd L. MANSFIELD, Cincinnati, OH (US); Robert H. TURNER, Cincinnati, OH (US)

(21) Appl. No.: 17/191,749

(22) Filed: Mar. 4, 2021

Related U.S. Application Data
(60) Provisional application No. 62/986,887, filed on Mar. 9, 2020.

(51) Int. Cl. A61F 13/49; C08L 91/00; A61F 13/15; B29C 65/48; B29C 65/00

(52) U.S. Cl. CPC A61F 13/4902; C08L 91/00; A61F 13/15699; A61F 13/15764; A61F 13/15211; B29C 65/48; B29L 2031/4878; A61F 2013/49025; A61F 2013/1591; A61F 2013/15235; A61F 2013/15292; A61F 13/49011; A61F 13/49014; B29C 66/7294

(57) ABSTRACT

The present disclosure relates to absorbent article incorporating elastomeric laminates that may be formed by stretching and joining an elastic strand with either or both first and second substrates using an adhesive. The elastic strands may be sourced from a wound supply, such as a beam of beamed elastic strands. The beam may be treated with a control layer to reducing blocking when the plurality of elastic strands are on the beam and to provide for reliable unwinding. The control layer may be absorbed into an adhesive once the elastic strands are joined with the first and second substrates with the adhesive to form the elastomeric laminate.

### CROSS REFERENCE TO RELATED APPLICATION

This application claims the benefit of U.S. Provisional Application No. 62/986,887, filed Mar. 9, 2020, which is incorporated herein by reference.

### FIELD OF THE INVENTION

The present disclosure relates to absorbent articles having elastomeric laminates, and more particularly, relates to the adhesives, control layer, and elastic strands of the elastomeric laminates.

### BACKGROUND OF THE INVENTION

Along an assembly line, various types of articles, such as for example, diapers and other absorbent articles, may be assembled by adding components to and/or otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. In some cases, individual components created from an advancing web or webs are combined with other individual components created from other advancing webs. Webs of material and component parts used to manufacture diapers may include: backsheets, topsheets, leg cuffs, waist bands, absorbent core components, front and/or back ears, fastening components, and various types of elastic webs and components such as leg elastics, barrier leg cuff elastics, stretch side panels, and waist elastics. Once the desired component parts are assembled, the advancing web(s) and component parts are subjected to a final knife cut to separate the web(s) into discrete diapers or other absorbent articles.

Some absorbent articles have components that include elastomeric laminates. Such elastomeric laminates may include an elastic material bonded to one or more nonwovens. The elastic material may include an elastic film and/or elastic strands. In some laminates, a plurality of elastic strands are joined to a nonwoven while the plurality of strands are in a stretched condition so that when the elastic strands relax, the nonwoven gathers between the locations where the nonwoven is bonded to the elastic strands, and in turn, forms corrugations. The resulting elastomeric laminate is stretchable to the extent that the corrugations allow the elastic strands to elongate.

In some assembly processes, stretched elastic strands may be advanced in a machine direction and adhered between two advancing substrates, wherein the stretched elastic strands are spaced apart from each other in a cross direction. Some assembly processes are also configured with several elastic strands that are very closely spaced apart from each other in the cross direction. In some configurations, close cross directional spacing between elastic strands can be achieved by drawing elastic strands from windings that have been stacked in the cross direction on a beam. For example, various textile manufacturers may utilize beam elastics and associated handling equipment, such as available from Karl Mayer Corporation.

However, problems can be encountered in manufacturing processes when using elastic strands stacked on a beam. For example, the elastic strands on the beam are prone to blocking when drawn from the beam due to cross-linking between the strands caused by the high compression of the beam over a substantial shelf life. To keep the elastic strands from blocking, they may be treated with a silicone oil or other type of spin finish. While applying a spin finish to the beam may reduce the likelihood of blocking, the spin finish may have undesired impacts on the manufacturing process. For example, when the elastic strands are formed into an elastomeric laminate using an adhesive to bond the strands to nonwovens layers, the spin finish may negatively impact the efficacy of the adhesive. In order to reach a desired level of adhesion, relatively large quantities of adhesive may be required. Using large quantities of adhesive is undesirable as cost of materials increase and also results in a stiff laminate that does not have the desired look or performance for incorporation into an absorbent article.

Consequently, it would be beneficial to provide a method and apparatus for producing elastomeric laminates from beams of elastic strands that utilize anti-blocking agents but can readily be adhered to nonwoven layers. It would further be beneficial to form a disposable absorbent article incorporating the elastomeric laminate.

### SUMMARY OF THE INVENTION

In a first aspect, a disposable absorbent article in the form of a diaper or absorbent pant may comprise a liquid permeable topsheet, a liquid impermeable backsheet, and an absorbent core disposed between the topsheet and the backsheet. The disposable absorbent article may comprise an elastomeric laminate. The elastomeric laminate may comprise a plurality of laterally-spaced elastic strands joined with a nonwoven web material by an adhesive. The elastic strands may comprise a strand polymer (e.g., segmented polyurethanes) wherein the strand polymer has a solubility parameter within the range of about 18 MPa“² to about 18.5 MPa”². The adhesive may comprise an adhesive polymer and the adhesive polymer has a solubility parameter within the range of about 16 MPa“² to about 17.5 MPa”². The elastic strands may be sourced from a wound supply of elastic strands. The wound supply of elastic strands may comprise a control layer having a solubility parameter within the range of about 15.5 MPa“² to about 16.5 MPa”² and a number average molecular weight within the range of about 0.6 kg/mol to about 1.5 kg/mol.

In another aspect, a disposable absorbent article in the form of a diaper or absorbent pant may comprise an elastomeric laminate. The elastomeric laminate comprising a plurality of laterally-spaced elastic strands joined with at least a first layer of nonwoven web material by an adhesive. The elastic strands comprise a first block copolymer of the spandex-type. The block copolymer may comprise a rubber block and a rigid block. The rubber block may be selected from a group consisting of polyethers, polyesters, and combinations thereof. The adhesive may comprise an adhesive polymer and the adhesive polymer may comprise a second block copolymer of the styrenic type. In some implementations the adhesive may include a tackifier. The second block copolymer may comprise a rubber block and the rubber block may be selected from a group consisting of polyisoprene, polybutadiene, polyisoprene-co-butadiene, and hydrogenated variants thereof. A control layer may be at least partially dispersed from the elastic strand to the adhesive.

In yet another aspect, a disposable absorbent article in the form of a diaper or absorbent pant may comprise an elastomeric laminate. The elastomeric laminate may comprise a plurality of laterally-spaced elastic strands joined with at least a first layer of nonwoven web material by an adhesive. The elastic strands may comprise a first block copolymer of the spandex-type. The block copolymer may comprise a rubber block and a rigid block; and the rubber block may be selected from a group consisting of polyethers, polyesters, and combinations thereof. The adhesive may comprise an adhesive polymer and the adhesive polymer may comprise a second block copolymer of the styrenic type. The second block copolymer may comprise a rubber block that may be selected from a group consisting of polyisoprene, polybutadiene, polyisoprene-co-butadiene, and hydrogenated variants thereof. The elastomeric laminate may comprise a soap.

In another aspect, a process for making an elastomeric laminate may comprises unwinding elastomeric strands coated with a control layer. The control layer may comprise a mineral oil. The process may further comprise bonding the elastomeric strands between first and second substrate layers to form an elastomeric laminate. The elastomeric strands may have an Average Strand Spacing from about 0.25 mm to about 4 mm and the Average Dtex of the elastomeric strands may be from about 10 to about 500.

In yet another aspect, a method for assembling an elastomeric laminate may comprise providing a first substrate and a second substrate. The method may further comprise advancing elastic strands in a machine direction. The elastic strands may be separated from each other in a cross direction. The method may further comprise applying adhesive to at least one of the elastic strands, the first substrate, and the second substrate and combining the elastic strands with a first substrate and a second substrate to form an elastomeric laminate. The method may further comprise dispersing a control layer from the elastic strands to the adhesive. The control layer may comprise a mineral oil.

### BRIEF DESCRIPTION OF THE DRAWINGS

FIG. 1A is a front perspective view of a diaper pant.

FIG. 1B is a rear perspective view of a diaper pant.

FIG. 2 is a partially cut away plan view of the diaper pant shown in FIGS. 1A and 1B in a flat, uncontracted state.

FIG. 3A is a cross-sectional view of the diaper pant of FIG. 2 taken along line 3A-3A.

FIG. 3B is a cross-sectional view of the diaper pant of FIG. 2 taken along line 3B-3B.

FIG. 4 is a schematic side view of a converting apparatus adapted to manufacture an elastomeric laminate including a first plurality of elastic strands positioned between a first substrate and a second substrate.

FIG. 5 is a view of the converting apparatus of FIG. 4 taken along line 5-5.

FIG. 6 shows an example of an empty beam.

FIG. 7 schematically depicts the adhering of the elastic strands to the first substrate.

FIG. 8 schematically depicts the interaction of a control layer with a plurality of elastic strands and an adhesive over time during the manufacture of an elastomeric laminate.

FIG. 9 illustrates a Laminate Creep Test.

FIG. 10 illustrates the Laminate Creep Test.

FIG. 11 illustrates the Laminate Creep Test.

### DETAILED DESCRIPTION OF THE INVENTION

The following term explanations may be useful in understanding the present disclosure:

“Absorbent article” is used herein to refer to consumer products whose primary function is to absorb and retain soils and wastes. “Diaper” is used herein to refer to an absorbent article generally worn by infants and incontinent persons about the lower torso. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

“Longitudinal” means a direction running substantially perpendicular from a waist edge to a longitudinally opposing waist edge of an absorbent article when the article is in a flat out, uncontracted state, or from a waist edge to the bottom of the crotch, i.e. the fold line, in a bi-folded article. Directions within 45 degrees of the longitudinal direction are considered to be “longitudinal.” “Lateral” refers to a direction running from a longitudinally extending side edge to a laterally opposing longitudinally extending side edge of an article and generally at a right angle to the longitudinal direction. Directions within 45 degrees of the lateral direction are considered to be “lateral.”

The term “substrate” is used herein to describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

The term “taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations in U.S. Pat. Nos. 5,167,897, 5,360,420, 5,599,335, 5,643,588, 5,674,216, 5,702,551, 5,968,025, 6,107,537, 6,118,041, 6,153,209, 6,410,129, 6,426,444, 6,586,652, 6,627,787, 6,617,016, 6,825,393, and 6,861,571; and U.S. Patent Publication Nos. 2013/0072887 A1; 2013/0211356 A1; and 2013/0306226 A1.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be pre-formed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be pre-formed anywhere along the circumference of the article in the waist region (e.g., side fastened or seamed, front waist fastened or seamed, rear waist fastened or seamed). Example diaper pants in various configurations are disclosed in U.S. Pat. Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1, 2012/0061016 A1, 2012/0061015 A1; 2013/0255861 A1; 2013/0255862 A1; 2013/0255863 A1; 2013/0255864 A1; and 2013/0255865 A1.

“Decitex” also known as “Dtex” is a measurement used in the textile industry for measuring yarns or filaments. 1 Decitex=1 gram per 10,000 meters. In other words, if 10,000 linear meters of a relaxed yarn or filament weights 500 grams that yarn or filament would have a decitex of 500.

The present disclosure relates to disposable absorbent articles, and in particular, to disposable absorbent articles incorporating elastomeric laminates as well as the processes for making the elastomeric laminates. Disposable absorbent articles according to the present disclosure may be in the form of a diaper or absorbent pant, comprising a liquid permeable topsheet, a liquid impermeable backsheet, and an absorbent core disposed between the topsheet and the backsheet. The disposable absorbent articles may also comprise an elastomeric laminate that comprises a plurality of laterally-spaced elastic strands joined with a nonwoven web material by an adhesive. The elastic strands may comprise a strand polymer that has a solubility parameter within the range of about 18 MPa“² to about 18.5 MPa”², for example. The adhesive may comprise an adhesive polymer (e.g., styrenic block copolymers or polyolefin-based polymers, or blends thereof) that has a solubility parameter within the range of about 16 MPa“² to about 17.5 MPa”²; for example. Adhesives of the present disclosure may or may not comprise a tackifier. Further, adhesives of the present disclosure may comprise less than 20% tackifier, less than 15% tackifier, less than 10%, or less than 5% tackifier. The elastic strands may be sourced from a wound supply of elastic strands, such as a beam, spool, or other supply source. The wound supply of elastic strands may comprise a control layer that has a solubility parameter within the range of about 15.5 MPa“² to about 16.5 MPa”² and a number average molecular weight within the range of about 0.6 kg/mol to about 1.5 kg/mol, for example. See CRC Handbook of Chemistry and Physics, 97th Edition,” CRC Press, Taylor & Francis Group, 6000 Broken Sound Parkway NW, Suite 300, Boca Raton, Fla. 33487-2742 for additional information regarding the determination of solubility parameters in accordance with the present disclosure. See Introduction to Polymers, 2^{nd} Edition, R. J. Young and P. A. Lovell, pages 211-221 for additional information regarding the determination of number average molecular weight (using polystyrene as the calibration standard and based on the refractive index (RI) detector) in accordance with the present disclosure.

The beam may comprise from about 40 to about 1000 elastic strands, or from about 100 to about 750 elastic strands, or from about 200 to about 600 elastic strands, or from about 300 to about 500 elastic strands. It should be understood that while the present disclosure emphasizes the benefits of using a control layer with a beam comprising many fine (less than about 500 decitex) elastic strands, it may also be desirable to use a control layer on a spool that may comprise a single elastic strand. Further, it may be desirable to use a control layer on traditionally sized elastic strands (greater than about 500 dtex).

Further, the elastomeric laminates according to the present disclosure may comprise a plurality of laterally-spaced elastic strands that comprise a spandex-type polymer. Commercially available Spandex strands may also be known as Lycra, Creora, Roica, or Dorlastan. Spandex polymers are sometimes referred to as Elastane, segmented polyurethane copolymers, or segmented polyurea copolymers. Spandex polymers contain rubber blocks and rigid blocks. These blocks are connected by urethane or urea chemical linkages. Typical rubber blocks include polyethers like polytetramethylene oxide or polyesters such as polycaprolactone. The rigid block may comprise diisocyanates such as diphenylmethane 4,4′-diisocyanate (MDI) and toluene-2,4-diisocyanate (TDI). These diisocyanates can optionally be coupled together using diols such as butanediol or diamines such as hydrazine or ethylene diamine. It's understood that a variety of rubber blocks, rigid blocks and coupling agents can be contemplated for use. For example, rubber block polymers can include polyesters such as polyethylene adipate, polypropylene adipate, and polybutylene adipate, poly-1,5-pentanediol, 1,6-hexanediol, or 1,10-decanediol or polyethers such as polyethylene glycol, polypropylene glycol, polytetramethylene glycol and the like. Similarly, rigid blocks can contain diphenylmethane 4,4′-diisocyanate (MDI), toluene-2,4-diisocyanate (TDI), hexamethylene diisocyanate (HDI), methylene dicyclohexyl diisocyanate (hydrogenated MDI (HMDI)) or isophorone diisocyanate (IPDI). Similarly, the optional coupling agents for the rigid block can include diamines (hydrazine and ethylene diamine, etc.) or diols (butane diol, 1,5-pentanediol, 1,6-hexanediol, etc.).

The adhesive of the elastomeric laminate may comprise an adhesive polymer that comprises a block copolymer of the styrenic type. The block copolymer may comprise a rubber block that is selected from a group consisting of polyisoprene, polybutadiene, polyisoprene-co-butadiene, and hydrogenated variants thereof. In some embodiments, the elastomeric laminate may also comprise a soap.

Further, adhesive polymers of the present disclosure can be styrenic block copolymers or polyolefin-based polymers, or blends thereof. Styrenic block copolymers of the present disclosure may include styrene-butadiene (SB), styrene-butadiene-styrene (SBS), styrene-isoprene-styrene (SIS), styrene-isoprene (SI), styrene-isoprene-butadiene-styrene (SIBS), styrene-ethylene-butylene-styrene (SEBS), styrene-ethylene-butylene (SEB) styrene-ethylene propylene-styrene (SEPS) and styrene-ethylene propylene (SEP) and styrene-ethylene-ethylene-propylene-styrene (SEEPS or hydrogenated SIBS). Styrenic block copolymers of the present disclosure may have the general configuration A-B-A or mixtures of A-B and A-B-A wherein the polymer end-blocks A are styrene while the polymer mid-block B is derived from isoprene, butadiene or isobutylene which may be partially or substantially hydrogenated or mixtures thereof. Further, the copolymers may be linear or branched. Notably, a styrene content of greater than 40% in the styrenic block copolymers may reinforce creep resistance while a melt flow index greater than 33 may enable a desirable viscosity. Polyolefin-based polymers of the present disclosure can be propylene homopolymers and propylene-based polymers that are copolymers with one or more other comonomers (e.g., ethylene, butene, pentene, octene, etc.). The propylene-based polymers can be based entirely on olefins, i.e. do not contain any functional groups. The propylene-based polymers can comprise greater than 75% by weight propylene or even greater than 80% by weight propylene. Further, the propylene-based polymers can comprise 10-20 mol % comonomer or 13-16 mol % comonomer. The propylene-based polymers can have a polydispersity (Mw/Mn) of less than about 5, less than about 3, or even about 2. Useful propylene-based polymers can have a density of no greater than about 0.90, no greater than about 0.89, or even no greater than about 0.88. Useful propylene-based polymers include single-site (e.g., metallocene) catalyzed propylene-based polymers. In addition, the polyolefin-based polymers can be copolymers of ethylene and C₃ to C₂₀-alpha-olefins prepared in the presence of metallocene as catalyst.

According to the present disclosure, a process for making an elastomeric laminate may comprise unwinding elastomeric strands that are coated with a control layer. The control layer may comprise, for example, a mineral oil, a paraffinic mineral oil, a white mineral oil, a synthetic oil, polyisoprene, and/or polybutadiene. The process may include bonding the elastomeric strands between first and second substrate layers to form an elastomeric laminate, with the elastomeric strands having an Average Strand Spacing from about 0.25 mm to about 4 mm, or from about 0.25 mm to about 3 mm, or from about 0.5 mm to about 3 mm, or from about 0.25 mm to about 2 mm or from about 0.5 mm to about 2 mm, for example. Further, the Average Dtex of the elastomeric strands may be in a range from about 10 to about 500, or from about 10 to about 400, or from about 10 to about 300. The relative quantity of control layer utilized on the elastomeric strands can vary, but in some embodiments the control layer is less than about 5%, or less than 3%, or less than 2% by weight of the elastomeric strands.

Additionally, according to the present disclosure, a method for assembling an elastomeric laminate may comprise providing a first substrate and a second substrate and advancing elastic strands in a machine direction. The elastic strands may be separated from each other in a cross direction. The method may also include applying adhesive to at least one of the elastic strands, the first substrate, and the second substrate and combining the elastic strands with the first substrate and the second substrate to form an elastomeric laminate. The method may also include dispersing a control layer that comprises mineral oil from the elastic strands to the adhesive.

As previously mentioned, the elastomeric laminates made according to the processes and apparatuses discussed herein may be used to construct various types of components used in the manufacture of different types of absorbent articles, such as diaper pants and taped diapers. To help provide additional context to the subsequent discussion of the process embodiments, the following provides a general description of absorbent articles in the form of diapers that include components including the elastomeric laminates that may be produced with the methods and apparatuses disclosed herein.

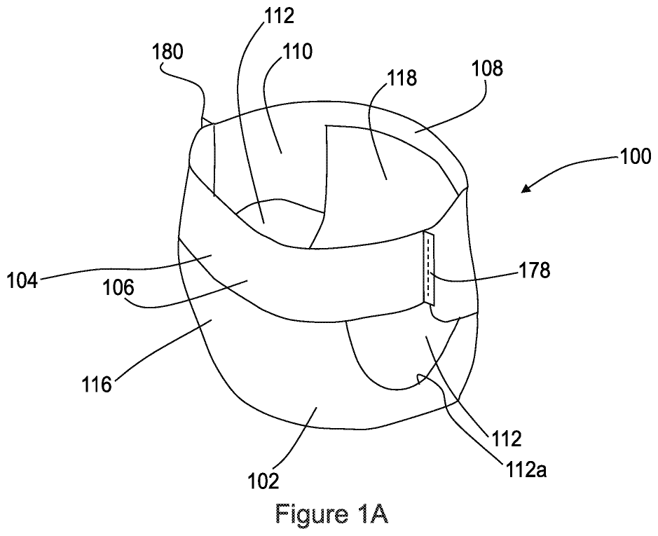

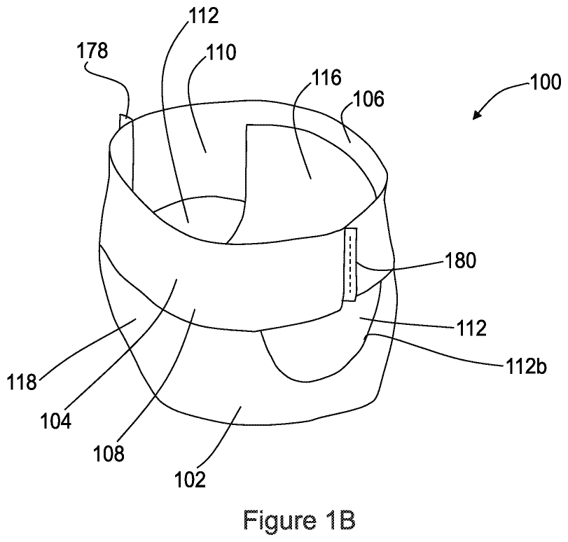

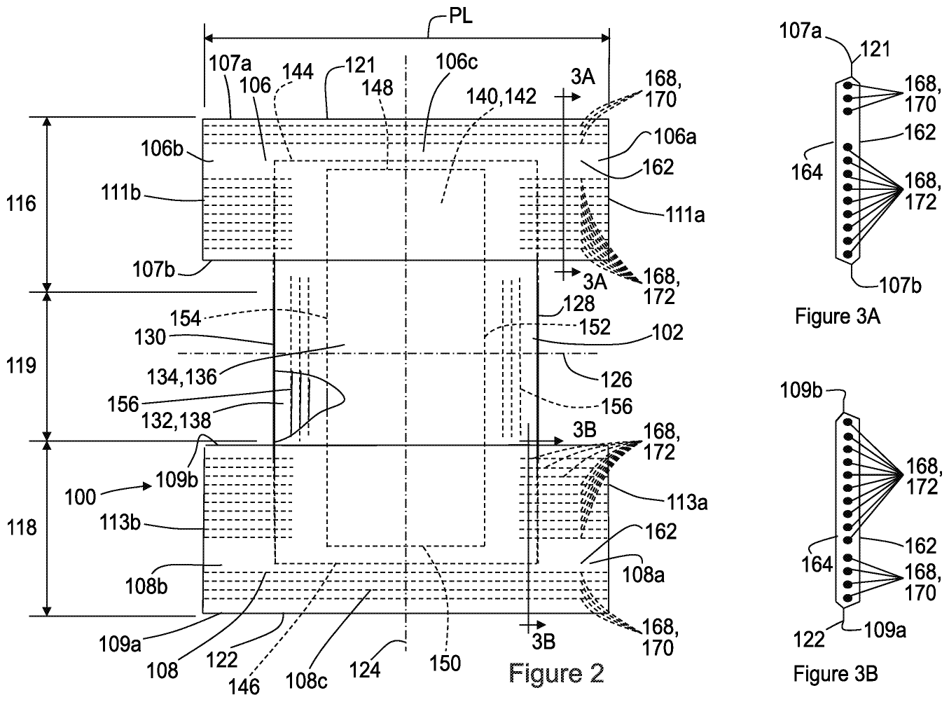

FIGS. 1A, 1B, and 2 show an example of a diaper pant 100 that may include components constructed from elastomeric laminates assembled in accordance with the apparatuses and methods disclosed herein. In particular, FIGS. 1A and 1B show perspective views of a diaper pant 100 in a pre-fastened configuration, and FIG. 2 shows a plan view of the diaper pant 100 with the portion of the diaper that faces away from a wearer oriented toward the viewer. The diaper pant 100 includes a chassis 102 and a ring-like elastic belt 104. As discussed below in more detail, a first elastic belt 106 and a second elastic belt 108 may be bonded together to form the ring-like elastic belt 104.

With continued reference to FIG. 2, the diaper pant 100 and the chassis 102 each include a first waist region 116, a second waist region 118, and a crotch region 119 disposed intermediate the first and second waist regions. The first waist region 116 may be configured as a front waist region, and the second waist region 118 may be configured as back waist region. The diaper 100 may also include a laterally extending front waist edge 121 in the front waist region 116 and a longitudinally opposing and laterally extending back waist edge 122 in the back waist region 118. To provide a frame of reference for the present discussion, the diaper 100 and chassis 102 of FIG. 2 are shown with a longitudinal axis 124 and a lateral axis 126. In some embodiments, the longitudinal axis 124 may extend through the front waist edge 121 and through the back waist edge 122. And the lateral axis 126 may extend through a first longitudinal or right side edge 128 and through a midpoint of a second longitudinal or left side edge 130 of the chassis 102.

As shown in FIGS. 1A, 1B, and 2, the diaper pant 100 may include an inner, body facing surface 132, and an outer, garment facing surface 134. The chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the diaper 100 may also include other features, such as leg elastics and/or leg cuffs to enhance the fit around the legs of the wearer.

As shown in FIG. 2, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 2, the laterally extending end edges 144 and 146 may be located longitudinally inward from the laterally extending front waist edge 121 in the front waist region 116 and the laterally extending back waist edge 122 in the back waist region 118. When the diaper pant 100 is worn on the lower torso of a wearer, the front waist edge 121 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

As previously mentioned, the diaper pant 100 may include a backsheet 136. The backsheet 136 may also define the outer surface 134 of the chassis 102. The backsheet 136 may also comprise a woven or nonwoven material, polymeric films such as thermoplastic films of polyethylene or polypropylene, and/or a multi-layer or composite materials comprising a film and a nonwoven material. The backsheet may also comprise an elastomeric film. An example backsheet 136 may be a polyethylene film having a thickness of from about 0.012 mm (0.5 mils) to about 0.051 mm (2.0 mils). Further, the backsheet 136 may permit vapors to escape from the absorbent core (i.e., the backsheet is breathable) while still preventing exudates from passing through the backsheet 136.

Also described above, the diaper pant 100 may include a topsheet 138. The topsheet 138 may also define all or part of the inner surface 132 of the chassis 102. The topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art. Topsheets 138 may be selected from high loft nonwoven topsheets, apertured film topsheets and apertured nonwoven topsheets. Exemplary apertured films may include those described in U.S. Pat. Nos. 5,628,097; 5,916,661; 6,545,197; and 6,107,539.

As mentioned above, the diaper pant 100 may also include an absorbent assembly 140 that is joined to the chassis 102. As shown in FIG. 2, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 A1 and 2004/0097895 A1.

As previously mentioned, the diaper 100 may also include elasticized leg cuffs 156. It is to be appreciated that the leg cuffs 156 can be and are sometimes also referred to as leg bands, side flaps, barrier cuffs, elastic cuffs or gasketing cuffs. The elasticized leg cuffs 156 may be configured in various ways to help reduce the leakage of body exudates in the leg regions. Example leg cuffs 156 may include those described in U.S. Pat. Nos. 3,860,003; 4,909,803; 4,695,278; 4,795,454; 4,704,115; 4,909,803; and U.S. Patent Publication No. 2009/0312730 A1.

Diaper pants may be manufactured with a ring-like elastic belt 104 and provided to consumers in a configuration wherein the front waist region 116 and the back waist region 118 are connected to each other as packaged, prior to being applied to the wearer. As such, diaper pants may have a continuous perimeter waist opening 110 and continuous perimeter leg openings 112 such as shown in FIGS. 1A and 1B. The ring-like elastic belt may be formed by joining a first elastic belt to a second elastic belt with a permanent side seam or with an openable and reclosable fastening system disposed at or adjacent the laterally opposing sides of the belts.

The ring-like elastic belt 104 may be defined by a first elastic belt 106 connected with a second elastic belt 108. As shown in FIG. 2, the first elastic belt 106 extends between a first longitudinal side edge 111a and a second longitudinal side edge 111b and defines first and second opposing end regions 106a, 106b and a central region 106c. And the second elastic 108 belt extends between a first longitudinal side edge 113a and a second longitudinal side edge 113b and defines first and second opposing end regions 108a, 108b and a central region 108c. The distance between the first longitudinal side edge 111a and the second longitudinal side edge 111b defines the pitch length, PL, of the first elastic belt 106, and the distance between the first longitudinal side edge 113a and the second longitudinal side edge 113b defines the pitch length, PL, of the second elastic belt 108. The central region 106c of the first elastic belt may be connected with the first waist region 116 of the chassis 102, and the central region 108c of the second elastic belt 108 may be connected with the second waist region 118 of the chassis 102. As shown in FIGS. 1A and 1B, the first end region 106a of the first elastic belt 106 may be connected with the first end region 108a of the second elastic belt 108 at first side seam 178, and the second end region 106b of the first elastic belt 106 may be connected with the second end region 108b of the second elastic belt 108 at second side seam 180 to define the ring-like elastic belt 104 as well as the waist opening 110 and leg openings 112.

As shown in FIGS. 2, 3A, and 3B, the first elastic belt 106 also defines an outer laterally extending edge 107a and an inner laterally extending edge 107b, and the second elastic belt 108 defines an outer laterally extending edge 109a and an inner laterally extending edge 109b. As such, a perimeter edge 112a of one leg opening may be defined by portions of the inner laterally extending edge 107b of the first elastic belt 106, the inner laterally extending edge 109b of the second elastic belt 108, and the first longitudinal or right side edge 128 of the chassis 102. And a perimeter edge 112b of the other leg opening may be defined by portions of the inner laterally extending edge 107b, the inner laterally extending edge 109b, and the second longitudinal or left side edge 130 of the chassis 102. The outer laterally extending edges 107a, 109a may also define the front waist edge 121 and the laterally extending back waist edge 122 of the diaper pant 100. The first elastic belt and the second elastic belt may also each include an outer, garment facing layer 162 and an inner, wearer facing layer 164. It is to be appreciated that the first elastic belt 106 and the second elastic belt 108 may comprise the same materials and/or may have the same structure. In some embodiments, the first elastic belt 106 and the second elastic belt may comprise different materials and/or may have different structures. It should also be appreciated that the first elastic belt 106 and the second elastic belt 108 may be constructed from various materials. For example, the first and second belts may be manufactured from materials such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some embodiments, the first and second elastic belts include a nonwoven web of synthetic fibers, and may include a stretchable nonwoven. In other embodiments, the first and second elastic belts include an inner hydrophobic, non-stretchable nonwoven material and an outer hydrophobic, non-stretchable nonwoven material.

The first and second elastic belts 106, 108 may also each include belt elastic material interposed between the outer substrate layer 162 and the inner substrate layer 164. The belt elastic material may include one or more elastic elements such as strands, ribbons, films, or panels extending along the lengths of the elastic belts. As shown in FIGS. 2, 3A, and 3B, the belt elastic material may include a plurality of elastic strands 168 which may be referred to herein as outer, waist elastics 170 and inner, waist elastics 172. Elastic strands 168, such as the outer waist elastics 170, may continuously extend laterally between the first and second opposing end regions 106a, 106b of the first elastic belt 106 and between the first and second opposing end regions 108a, 108b of the second elastic belt 108. In some embodiments, some elastic strands 168, such as the inner waist elastics 172, may be configured with discontinuities in areas, such as for example, where the first and second elastic belts 106, 108 overlap the absorbent assembly 140. In some embodiments, the elastic strands 168 may be disposed at a constant interval in the longitudinal direction. In other embodiments, the elastic strands 168 may be disposed at different intervals in the longitudinal direction. The belt elastic material in a stretched condition may be interposed and joined between the uncontracted outer layer and the uncontracted inner layer. When the belt elastic material is relaxed, the belt elastic material returns to an unstretched condition and contracts the outer layer and the inner layer. The belt elastic material may provide a desired variation of contraction force in the area of the ring-like elastic belt. It is to be appreciated that the chassis 102 and elastic belts 106, 108 may be configured in different ways other than as depicted in FIG. 2. The belt elastic material may be joined to the outer and/or inner layers continuously or intermittently along the interface between the belt elastic material and the inner and/or outer belt layers.

In some configurations, the first elastic belt 106 and/or second elastic belt 108 may define curved contours. For example, the inner lateral edges 107b, 109b of the first and/or second elastic belts 106, 108 may include non-linear or curved portions in the first and second opposing end regions. Such curved contours may help define desired shapes to leg opening 112, such as for example, relatively rounded leg openings. In addition to having curved contours, the elastic belts 106, 108 may include elastic strands 168, 172 that extend along non-linear or curved paths that may correspond with the curved contours of the inner lateral edges 107b, 109b.

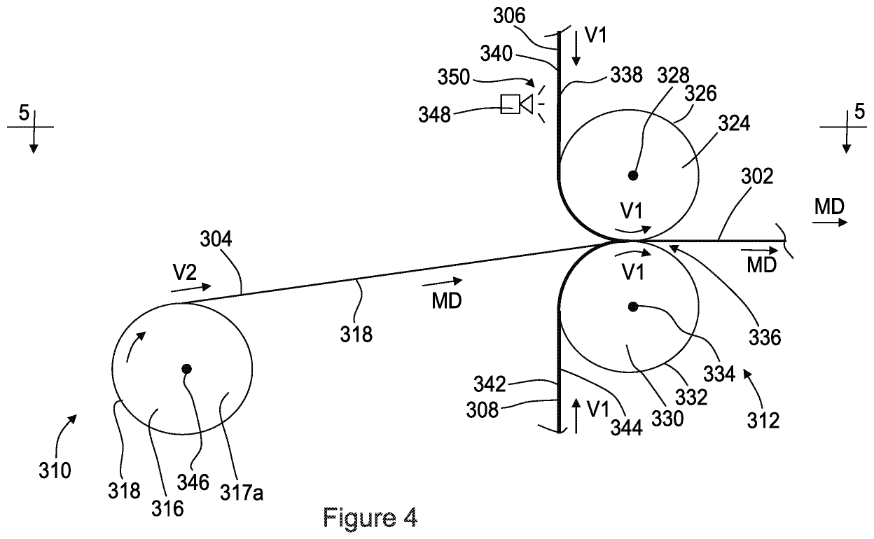

Apparatuses and methods according to the present disclosure may be utilized to produce elastomeric laminates that may be used to construct various components of diapers, such as elastic belts, leg cuffs, and the like. For example, FIGS. 4-8 show various schematic views of converting apparatuses 300 adapted to manufacture elastomeric laminates 302. As described in more detail below, the converting apparatuses 300 shown in FIGS. 4-8 operate to advance a continuous length of elastic material 304, a continuous length of a first substrate 306, and a continuous length of a second substrate 308 along a machine direction MD. It is also to be appreciated that in some configurations, the first substrate and second substrate 306, 308 herein may be defined by two discrete substrates or may be defined by folded portions of a single substrate. The apparatus 300 may stretch the elastic material 304 and join the stretched elastic material 304 with the first and second substrates 306, 308 to produce an elastomeric laminate 302. The elastic material 304 may be sourced from a rotating beam of elastic strands wound thereon, or other type of wound supply of elastic strands. During operation, elastic material may advance in a machine direction from the rotating beam.

The elastomeric laminates 302 can be used to construct various types of diaper components, such as the belts, ear panels, side panels, transverse barriers, topsheets, backsheets, cuffs, waistbands, waistcaps, and/or chassis. For example, the elastomeric laminates 302 may be used as a continuous length of elastomeric belt material that may be converted into the first and second elastic belts 106, 108 discussed above with reference to FIGS. 1-3B. As such, the elastic material 304 may correspond with the belt elastic material 168 interposed between the outer layer 162 and the inner layer 164, which in turn, may correspond with either the first and/or second substrates 306, 308. In other examples, the elastomeric laminates may be used to construct waistbands and/or side panels in taped diaper configurations. In yet other examples, the elastomeric laminates may be used to construct various types of leg cuff and/or topsheet configurations. When the elastomeric laminate 302 forms at least a portion of at least one of the group consisting of a belt, a chassis, a side panel, a topsheet, a backsheet, and an ear panel, and combinations thereof, the plurality of elastics 318 of the elastomeric laminate 302 may comprise from about 40 to about 1000 elastic strands. And, when the elastomeric laminate 302 forms at least a portion of at least one of the group consisting of a waistband, a waistcap, an inner leg cuff, an outer leg cuff, and combinations thereof, the first plurality of elastics 316 of the elastomeric laminate 302 may comprise from about 10 to about 400 elastic strands. Ultimately, “plurality of elastics” is a term of context, where certain properties, arrangements, attributes, characteristics, disposition, etc. of the elastics are referenced to define what a certain “plurality of elastics” is.

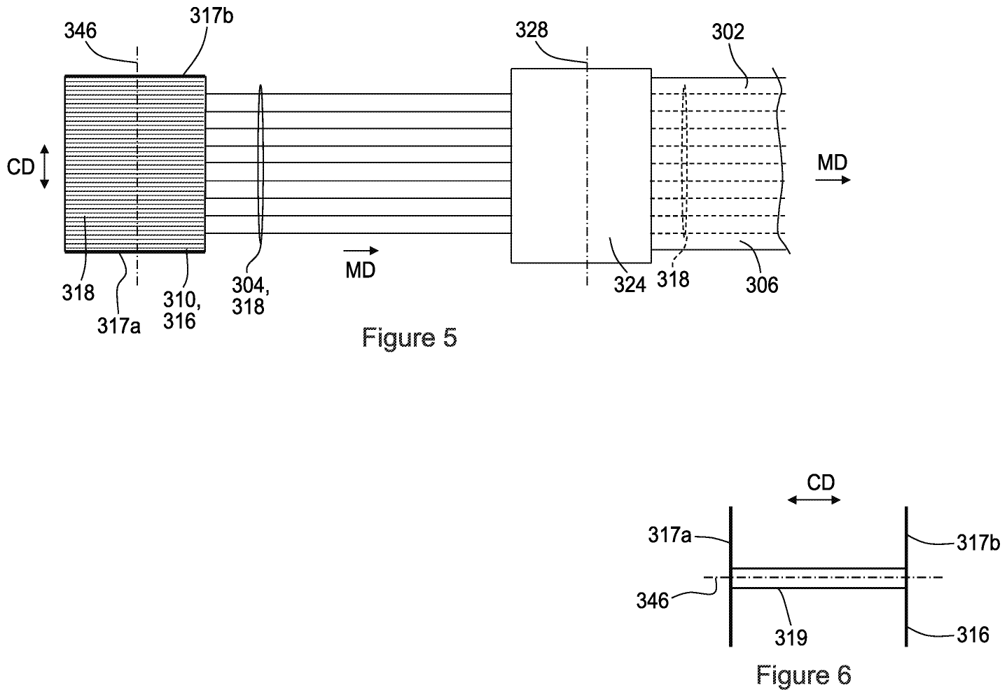

As shown in FIGS. 4-5, a converting apparatus 300 for producing an elastomeric laminate 302 may include a first metering device 310 and a second metering device 312. The first metering device may be configured as a beam 316 with a plurality of elastic strands 318 wound thereon. FIG. 6 shows an example of an empty beam 316 that includes two side plates 317a, 317b that may be connected with opposing end portions of a mandrel core 319, wherein elastic strands may be wound onto the mandrel core 319. It is to be appreciated that beams of various sizes and technical specifications may be utilized in accordance with the methods and apparatuses herein, such as for example, beams that are available from ALUCOLOR Textilmaschinen, GmbH. During operation, the plurality of elastic strands 318 advance in the machine direction MD from the beam 316 to the second metering device 312. In addition, the plurality of elastic strands 318 may be stretched along the machine direction MD between the beam 316 and the second metering device 312. The stretched elastic strands 318 may be joined with a first substrate 306 and a second substrate 308 at the second metering device 312 to produce an elastomeric laminate 302. It is noted, however, that in some configurations the elastic strands 318 are not arranged in a beam format. Instead, for example, the first metering device 310 may be a spool of an individual elastic strand 318, or otherwise be a spool of elastic strands 318 that are not formed into a beam. As such, the systems and methods described herein are applicable across a range of manufacturing processes that generally seek to adhere one or more elastic strands to one or more substrates.

As shown in FIG. 4, the second metering device 312 includes: a first roller 324 having an outer circumferential surface 326 and rotates about a first axis of rotation 328, and a second roller 330 having an outer circumferential surface 332 and rotates about a second axis of rotation 334. The first roller 324 and the second roller 330 rotate in opposite directions, and the first roller 324 may be adjacent the second roller 330 to define a nip 336 between the first roller 324 and the second roller 330. The first roller 324 rotates such that the outer circumferential surface 326 has a surface speed V1, and the second roller 330 may rotate such that the outer circumferential surface 332 has the same, or substantially the same, surface speed V1.

As shown in FIGS. 4 and 5, the first substrate 306 includes a first surface 338 and an opposing second surface 340, and the first substrate 306 advances to the first roller 324. In particular, the first substrate 306 advances at speed V1 to the first roller 324 where the first substrate 306 partially wraps around the outer circumferential surface 326 of the first roller 324 and advances through the nip 336. As such, the first surface 338 of the first substrate 306 travels in the same direction as and in contact with the outer circumferential surface 326 of the first roller 324. In addition, the second substrate 308 includes a first surface 342 and an opposing second surface 344, and the second substrate 308 advances to the second roller 330. In particular, the second substrate 308 advances at speed V1 to the second roller 330 where the second substrate 308 partially wraps around the outer circumferential surface 332 of the second roller 330 and advances through the nip 336. As such, the second surface 344 of the second substrate 308 travels in the same direction as and in contact with the outer circumferential surface 332 of the second roller 330.

With continued reference to FIGS. 4 and 5, the beam 316 includes the plurality of elastic strands 318 wound thereon, and the beam 316 is rotatable about a first beam rotation axis 346. In some configurations, the first beam rotation axis 346 may extend in the cross direction CD. As the beam 316 rotates, the plurality of elastic strands 318 advance from the beam 316 at a speed V2 with the plurality of elastic strands 318 being spaced apart from each other in the cross direction CD from about 0.25 mm to about 4 mm, or from about 0.25 mm to about 3 mm, or from about 0.25 mm to about 2 mm From the beam 316, the plurality of elastic strands 318 advances in the machine direction MD to the nip 336. In some configurations, the speed V2 is less than the speed V1, and as such, the plurality of elastic strands 318 may be stretched in the machine direction MD. In turn, the stretched plurality of elastic strands 318 advance through the nip 336 between the first and second substrates 306, 308 such that the plurality of elastic strands 318 may be joined with the second surface 340 of the first substrate 306 and the first surface 342 of the second substrate 308 to produce a continuous length of elastomeric laminate 302. As shown in FIG. 4, the first substrate 306 may advance past an adhesive applicator device 348 that applies adhesive 350 to the second surface 340 of the first substrate 306 before advancing to the nip 336. It is to be appreciated that the adhesive 350 may be applied to the first substrate 306 upstream of the first roller 324 and/or while the first substrate 306 is partially wrapped around the outer circumferential surface 326 of the first roller 324. It is to be appreciated that adhesive may be applied to the plurality of elastic strands 318 before and/or while being joined with first substrate 306 and second substrate 308. In addition, it is to be appreciated that adhesive may be applied to the first surface 342 of the second substrate 308 before or while being joined with the plurality of elastic strands 318 and the first substrate 306.

It is to be appreciated that different components may be used to construct the elastomeric laminates 302 in accordance with the methods and apparatuses herein. For example, the first and/or second substrates 306, 308 may include nonwovens and/or films. In addition, the plurality of elastic strands 318 may be configured in various ways and having various decitex values. In some configurations, the plurality of elastic strands 318 may be configured with decitex values ranging from about 10 decitex to about 500 decitex, or from about 10 decitex to about 400 decitex, or from about 10 decitex to about 300 decitex, specifically reciting all 1 decitex increments within the above-recited ranges and all ranges formed therein or thereby. It is also to be appreciated the beam 316 may be configured in various ways and with various quantities of elastic strands. Example beams, also referred to as warp beams, that may be used with the apparatus and methods herein are disclosed in U.S. Pat. Nos. 4,525,905; 5,060,881; and 5,775,380; and U.S. Patent Publication No. 2004/0219854 A1. Although FIG. 5 shows nine elastic strands 318 advancing from the beam 316, it is to be appreciated that the apparatuses herein may be configured such that more or less than nine elastic strands 318 advance from the beam 316. In some configurations, the plurality of elastic strands 318 advancing from the beam 316 may include from about 100 to about 2000 strands, specifically reciting all 1 strand increments within the above-recited range and all ranges formed therein or thereby. In some configurations, the plurality of elastic strands 318 may be separated from each other by about 0.5 mm to about 4 mm in the cross direction, specifically reciting all 0.1 mm increments within the above-recited range and all ranges formed therein or thereby. The elastics in the plurality of elastic strands may be pre-strained prior to joining the elastic strand to the first or second substrate layers 306, 308. In some configurations, the elastic may be pre-strained from about 75% to about 300%, specifically reciting all 1% increments within the above-recited range and all ranges formed therein or thereby. It is also to be appreciated that one or more beams of elastics may be arranged along the cross direction CD of a converting process and/or arranged along a machine direction MD in various different portions of a converting process. It is also to be appreciated that the beam 316 can be connected with one or more motors, such as servo motors, to drive and control the rotation of the beam 316.

It is also to be appreciated that the plurality of elastic strands 318 may have various different material constructions and/or decitex values to create elastomeric laminates 302 having different stretch characteristics in different regions. In some configurations, the elastomeric laminate may have regions where the elastic strands are spaced relatively close to one another in the cross direction CD and other regions where the elastic strands are spaced relatively farther apart from each other in the cross direction CD to create different stretch characteristics in different regions. In some configurations, the elastic strands may be supplied on the beam in a stretched state, and as such, may not require additional stretching (or may require relatively less additional stretching) before being combined with the first substrate 306 and/or the second substrate 308.

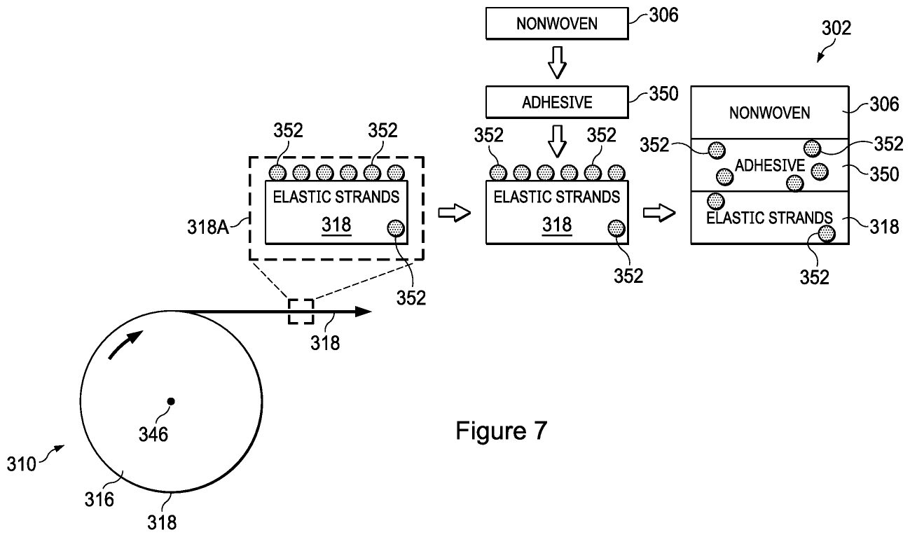

Referring now to FIG. 7, the adhering of the elastic strands 318 to the first substrate 306 is schematically depicted. As mentioned above, elastic strands that are wound around in a beam configuration under high compression are prone to stick (i.e. cross-link) during unwinding. As such, in accordance with the present disclosure, a control layer 352 can be applied to the elastic strands 318 of beam 316 to reducing cross-linking and to aid in the unwinding process. Moreover, as the elastic strands 318 may be adhered to the first and second substrate layers 306, 308 via an adhesive 350 (note that only first substrate 306 is shown in FIG. 7 for the purposes of illustration), various characteristics of the control layer 352 and the adhesive 350 can be specially selected as to allow for sufficient adhesion of the elastic strands 318 and the first and second substrate layers 306, 308. More particularly, the control layer 352 that is applied to the beam 316 can have a solubility level to ensure that a majority of the control layer 352 remains on the surface of the plurality of elastic strands 318, as opposed to being absorbed into the strands. This characteristic of the control layer 352 is schematically illustrated by the enlarged view 318A in FIG. 7. Thus, when the plurality of elastic strands 318 are drawn from the beam 316, undesirable blocking can be reduced or eliminated, even subsequent to the beam 316 being stored for a period of time under high compression prior to the unwinding of the elastic strands 318.

Importantly, however, the plurality of elastic strands 318 also need to be sufficiently adhered to the first and second substrate layers 306, 308 to form the elastomeric laminate 302 having the desired strength parameters. As such, the adhesive 350 can be specially selected to absorb the control layer 352 so that the control layer 352 does not negatively impact the adhesion of the adhesive 350 to the plurality of elastic strands 318. Thus, in accordance with the present disclosure, the plurality of elastic strands 318, the control layer 352 applied to the plurality of elastic strands 318, and the adhesive 350 are each specially selected so that the control layer 352 is not prone to be absorbed by the plurality of elastic strands 318 but, instead, is prone to be absorbed by the adhesive 350. As a result, the elastic strands 318 can be drawn from the beam 316 without blocking and the adhesive 350 can sufficiently adhere the elastic strands 318 and the first and second substrates 306, 308.

The desired level of partitioning in the elastomeric laminate 302 may be achieved by selecting a control layer 352 with a particular solubility parameter and average molecular weight. In particular, the control layer 352 may have a solubility parameter within the range of about 15.5 MPa^{1/2} to about 16.5 MPa^{1/2}, or about 15.8 MPa^{1/2} to about 16.5 MPa^{1/2} and a number average molecular weight within the range of about 0.6 kg/mol to about 1.5 kg/mol, or of about 0.8 kg/mol to about 1.4 kg/mol, or about 1.0 kg/mol to about 1.3 kg/mol. The control layer 352 may have a surface tension of about 24 mN/m to about 30 mN/m. Further, the adhesive 350 may comprise an adhesive polymer that has a solubility parameter within the range of about 16 MPa^{1/2} to about 17.5 MPa^{1/2}, or about 16.5 MPa^{1/2} to about 17.2 MPa^{1/2}. The plurality of elastic strands 318 may comprise a strand polymer that has a solubility parameter within the range of about 18 MPa^{1/2} to about 18.5 MPa^{1/2}. In accordance with one embodiment, the strand polymer has a solubility parameter of about 18.3 MPa^{1/2}. Furthermore, the number average molecular weight of the control layer may be lower than the molecular weight of each of the strand polymer of the plurality of elastic strands 318 and the adhesive polymer of the adhesive 350.

For a pair of materials a and b, the value associated with their mixing is calculated as:

$$χ = \frac{{υ ( {δ}_{a} - {δ}_{b} )}^{2}}{k T}$$

where:

k is 1.38 e-23 J/K (Boltzmann constant);

υ is the volume of the repeat unit of the higher molecular weight component in cubic meters;

T is the temperature in Kelvin; and

δ values are the solubility parameters of the components a and b in (MPa)\hat{ }0.5.

In accordance with the present disclosure, the χN value between the control layer 352 and the strand polymer of the elastic strands 318 is greater than 2, where N is a degree of polymerization of the control layer and x is the Flory-Huggins interaction parameter (see The Physics of Polymers, Gert R. Strobl, ISBN 978-3-642-06449-4 for additional information regarding the determination of χN in accordance with the present disclosure). Additionally, the χN value between the control layer 352 and the adhesive polymer of the adhesive 350 may be less than about 3, or less than about 2. The adhesive 350 may have a rubbery plateau modulus of elasticity of about 0.01 to about 0.3 MPa at 38° C. and 1 Hz, 0.02 to about 0.1 MPa at 38° C. and 1 Hz, or about 0.1 MPa at 38° C. and 1 Hz (see Pocious A. V., Adhesion and Adhesives Technology—an introduction, 2^{nd} Edition. Hanser/Gardner Publications, Inc., Cincinnati, Ohio (2002). ISBN 1-56990-319-0, pages 124-131 for additional information regarding the determination of plateau modulus in accordance with the present disclosure). Additionally, tensile modulus of the plurality of elastic strands 318 at room temperature may be within the range of about 5 MPa to about 15 MPa (see Pocious A. V., Adhesion and Adhesives Technology—an introduction, 2^{nd} Edition. Hanser/Gardner Publications, Inc., Cincinnati, Ohio (2002). ISBN 1-56990-319-0, pages 17-18 for additional information regarding the determination of tensile modulus in accordance with the present disclosure). Subsequent to the formation of the elastomeric laminate 302, the control layer 352 may disperse from its original location on the surface of the elastic strands 318 as it is absorbed into the adhesive 350.

As indicated above, beamed elastics in accordance with the present disclosure may be formed from Spandex fibers. One type of Spandex fiber is “PolyUrethane Urea” elastomer or the “high hard segment level PolyUrethane” elastomer, which may be formed into fibers using a solution (solvent) spinning process (as opposed to being processable in the molten state.) The rigid blocks in PolyUrethane Urea provides strong mutual chemical interactions crucial for providing “anchoring” that enables good stress relaxation performance at temperatures near body temperature on timescales corresponding to diaper wear, including overnight. This type of anchoring enables better Force Relaxation Over Time (i.e., little force decay with time when held in stretched condition at body temperature). In contrast, extruded strands and scrims are typically made of styrenic block copolymers or thermoplastic elastomers that can be formed in the molten state by conventional extrusion processes. Thermoplastic elastomers include compositions like polyolefin, polyurethane (PolyUrethane with hard segment melting below 200 deg. C.) elastomers, etc. Because these thermoplastic elastomers like Polyurethane (PolyUrethane with hard segment melting below 200 deg. C.) can be melted/remelted, and extruded it makes them susceptible to higher stress relaxation in use, which is a major negative. The styrenic block copolymers used in extruded strands comprise a comparatively long rubbery midblock situated between comparatively short end blocks. End blocks sufficiently short to enable good flow conventional extrusion processes often have a greater propensity to stress relax and undergo Force Relaxation Over Time. The Urea linkage present in Spandex requires it to be made by spinning process. Spandex cannot be melted/remelted or extruded like styrenic block copolymers. Spandex pre-polymer is combined with solvent and additives, and the solution is spun to make solid spandex fiber. Multiple fibers may then be formed together to make one spandex strand. One spandex fiber may have a decitex of about 15, so a 500 decitex strand may have nominally 33 fibers wound together to make one strand. Depending on the decitex used for beam approach, there may be 40 fibers (or filaments), 30 fibers, 20 fibers, 15 fibers, 8 fibers, 5 fibers, 3 fibers or even as low as 2 fibers. Spandex fiber can be mono-component or bi-component (as disclosed in WO201045637A2).

Commercially available Spandex strands may also be known as Lycra, Creora, Roica, or Dorlastan. Spandex is often referred as Elastan fiber or Polyurethane fiber. LYCRA HYFIT strands, a product of Invista, Wichita, Kans., are a suitable for making the strands that make up the plurality of elastics 318 that make up the elastomeric laminate 302. Some strands, for example, the aforementioned LYCRA HYFIT, may comprise a number of individual fibers wound together to form the strand. With regard to elastic strands formed of a number of individual fibers it has been discovered that the individual fibers can move relative to each other changing the cross-sectional shape of the strand as well as becoming unraveled which can lead to poor control of the strands as well as poor bonding/adhering/joining of the elastic strands to one or both of the first substrate layer 306 and second substrate layer 308 of the elastomeric laminate 302. In order to minimize the negatives with regard to strands comprising a plurality of fibers it would be advantageous to minimize the number of fibers in a given strand. It would therefore be desirable to have less than about 40 fibers per strand, less than about 30 fibers per strand, less than about 20 fibers per strand, less than about 10 fibers per strand, less than about 5 fibers per strand and 1 fiber forming the strand. In the case of a single fiber forming the strand which can deliver comparable performance to the multi-fiber strands of the prior art it would be desirable for the fiber to have a fiber decitex from about 22 to about 300 and a fiber diameter from about 50 micrometers to about 185 micrometers.

As provided above, the control layer 352 helps to prevent blocking when the plurality of elastic strands 318 are wound on a spool or a beam and it also lowers the coefficient of friction for the strands. In accordance with some embodiments, the control layer 352 is a mineral oil, which may be a paraffinic mineral oil, for example. In accordance with various embodiments, the control layer 352 may comprise any of white mineral oil, polyisoprene, and polybutadiene, for example. In other embodiments, the control layer may be a synthetic oil.

The control layer 352 may comprise additional materials to aid in its performance, such as a soap (i.e., a fatty acid or a fatty acid salt), a wax, a detergent, a clay, or an anti-caking agent (e.g., silica). The use of soap in accordance with the present disclosure is believed to reduce the tackiness of the elastic strands, which can improve handling in the winding process. Moreover, the use of a soap can also provide a beneficial tradeoff between unwindability and adherability.

In some implementations, for instance, a metallic soap may be added to the control layer 352 that serves to improve the unwindability of the plurality of elastic strands 318 from the beam 316. As used herein, metallic soap can be fatty acid salts that are fabricated by the reaction of alkaline, alkaline earth, or transition metals with saturated, unsaturated straight chain or branched aliphatic carboxylic acids with 8-22 carbon atoms, or 12-18 carbon atoms. Examples include saturated fatty acids, such as stearic acid (octadecanoic acid), lauric acid (dodecanoic acid), 12-hydroxystearic acid, and mixtures of acids with 8-22 carbon atoms; unsaturated fatty acids, such as oleic acid (cis-9-octadecenoic acid) and linoleic acid (9,12-octadecadienoic acid), synthetic carboxylic acids, such as isostearic acid, 2-ethylhexanoic acid, dimethylhexanoic acids, trimethylhexanoic acids; and mixtures of synthetic aliphatic isocarboxylic acids, and salts of the alicyclic naphthenic acids and resin acids. While a variety of metal ions can be used to make metallic soaps, examples include sodium, magnesium, calcium, and zinc. In accordance with one embodiment of the present disclosure, magnesium stearate is used as it is not soluble in the adhesive 350 nor the elastic strands 318. The quantity of soap utilized can vary, but in some embodiments the control layer 352 comprises about 1% to 5% by weight of soap, or from about 2% to 4% by weight of soap, or about 3% by weight of soap.

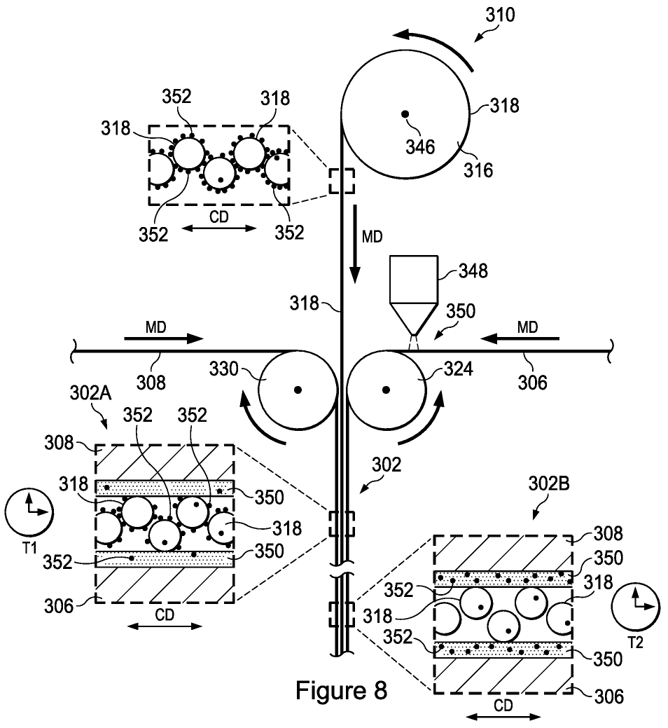

Referring now to FIG. 8, the interaction of the control layer 352 with the plurality of elastic strands 318 and the adhesive 350 over time is schematically illustrated. As illustrated by enlarged view 318A, the control layer 352 is schematically shown to generally coat the outer surface of the elastic strands 318. With the control layer 352 has a high molecular weight, it may not be materially absorbed by the elastic strands 318, even when the beam 316 is stored under high compression for a long period of time. As such, the control layer 352 serves to beneficially inhibit cross-linking and blocking when the elastic strands 318 are eventually drawn from the beam 316 during a manufacturing process. As previously described with reference to FIG. 4, the converting apparatus 300 produces the elastomeric laminate 302 that is formed by the first substrate 306, the plurality of elastic strands 318, and the second substrate 308. As shown in FIG. 8, the adhesive 350 may be used to adhere the plurality of elastic strands 318 to first and second substrates 306, 308. At a first point in time, denoted as time T1 in FIG. 8, the control layer 352 has begun to disperse in the elastomeric laminate 302A. In particular, due to the relative solubilities and number average molecular weights of the various components of the elastomeric laminate 302, as described above, the control layer 352 of the present disclosure may be absorbed mainly into the adhesive 350. With the control layer 352 absorbed into the adhesive 350, the adhesive 350 can adequately adhere to the elastic strands 318. Eventually, at a second point in time, denoted as time T2 in FIG. 8, the control layer 352 has completed the dispersion and may be substantially absorbed into the adhesive 350. Further, at T2, when the control layer 352 comprises a soap, the soap will not disperse into the adhesive 350. Instead, the soap will largely remain between the interface of the elastic strands 318 and the adhesive 350. For this reason, when excess soap is used, it can compromise adhesion of the first and second substrates 306,308 to the elastic strands 318. As shown, in FIG. 8 the adhesive 350 may contact a portion of the elastic strands 318. Alternatively, the adhesive 350 may substantially or completely wrap one or more of the elastic strands 318.

Consistent with what is generally stated above, desirably, use of the control layer 352 with the plurality of elastic strands 318 and the adhesive 350 to form an elastomeric laminate 302 will not yield materially different laminate properties than the same elastomeric laminate 302 made without the control layer 352. For instance, elastomeric laminates of the present disclosure that comprise a control layer may have a Laminate Creep of 5 mm or less, of 4 mm or less, or 3 mm or less according to the Laminate Creep Test. These Laminate Creep values are for laminates made with elastics comprising a control layer and evidence that the control layer of the present disclosure will not impact the performance of the adhesive. Further, elastomeric laminates of the present disclosure that is formed with elastic strands comprising a control layer may have a Laminate Creep that is within 2 mm or within 1 mm of the same elastomeric laminate that is formed with elastic strands that did not comprise a control layer—in fact, the elastomeric laminate that comprises the control layer may have a Laminate Creep that is less (i.e., less creep) than the same elastic laminate that does not comprise a control layer.

Further, elastomeric laminates of the present disclosure that comprise a control layer may have a Static Peel Force Time of greater than 700 min/10 mm bond length, greater than 600 min/10 mm bond length, greater than 500 min/10 mm bond length, or greater than 400 min/10 mm bond length, or greater than 300 min/10 mm bond length according to the Static Peel Force Time Test Method. These Static Peel Force Time values are for laminates made with elastics comprising a control layer and evidence that the control layer of the present disclosure will not impact the performance of the adhesive. Further, elastomeric laminates of the present disclosure that is formed with elastic strands comprising a control layer may have a Static Peel Force Time that is within 5 min/10 mm bond length or within 3 min/10 mm bond length of the same elastomeric laminate that is formed with elastic strands that did not comprise a control layer—in fact, the elastomeric laminate that comprises the control layer may have a Static Peel Force Time that is longer (i.e., more time) than the same elastic laminate that does not comprise a control layer.

Further, elastomeric laminates of the present disclosure that comprise a control layer may have a Force Relaxation Over Time of from about 5% to about 30%, from about 5% to about 25%, from about 10% to about 25%, or from about 15% to about 20% according to the Force Relaxation Over Time Method. These Force Relaxation Over Time values are for laminates made with elastics comprising a control layer and evidence that the control layer of the present disclosure will not impact the performance of the adhesive. Further, elastomeric laminates of the present disclosure that is formed with elastic strands comprising a control layer may have a Force Relaxation Over Time that is within 15% or within 10% of the same elastomeric laminate that is formed with elastic strands that did not comprise a control layer—in fact, the elastomeric laminate that comprises the control layer may have a Force Relaxation Over Time that is less (i.e., less force relaxation) than the same elastic laminate that does not comprise a control layer. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

Test Procedures

Unless otherwise noted, the tests are carried out under standard laboratory conditions of 22° C. and 50% relative humidity.

Force Relaxation Over Time

The Force Relaxation Over Time of a specimen is measured on a constant rate of extension tensile tester (a suitable instrument is the MTS Insight using Testworks 4.0 Software, as available from MTS Systems Corp., Eden Prairie, Minn.) using a load cell for which the forces measured are within 1% to 90% of the limit of the cell. Articles are conditioned at 23° C.±2 C.° and 50%±2% relative humidity for 2 hours prior to analysis and then tested under the same environmental conditions. Prepare a sample size such that it enables a gauge length of 25.4 mm (parallel to the elastic stretch) at a width of 12.7 mm.

Program the tensile tester to perform an elongation to determine the engineering strain at which the tensile force reaches 0.0294 N/mm.

Prepare and condition a second sample as described above for the Force Relaxation Over Time test. The test is performed on the same equipment as described above. It is performed at a temperature of 37.8° C. Extend the sample to the strain as determined above. Hold the sample for 10 hours and record the force at a rate of at least 5 Hz during application of the strain, at least 5 Hz for the first minute of force relaxation, and at least 0.05 Hz (one point every 20 seconds) thereafter throughout the experiment.

Laminate Creep Test Method (“Laminate Creep”)

The Laminate Creep Test Method is used to characterize the movement of the ends of stretched elastic strands 318 of a stretched elastomeric laminate away from a cut edge 472 of the same laminate.

Apparatus

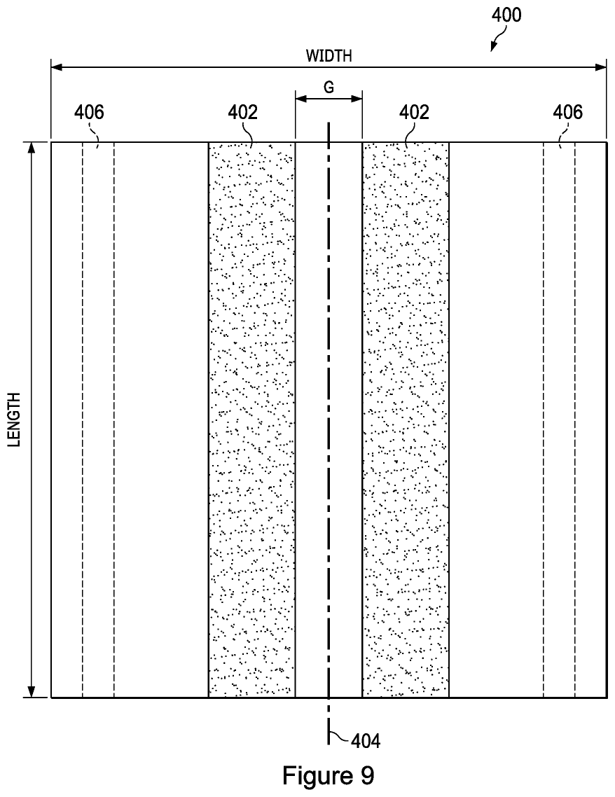

A stretch board 400 is prepared from acrylic or polycarbonate sheet, an example of which is shown in FIG. 9. The width of the stretch board 400 is between 250 and 375 mm, and the length of the stretch board 400 is at least as long as the laminate to be tested is wide. Hook material 402 that is capable of securing the elastomeric laminate is affixed to the front side of the stretch board 400, running in the lengthwise direction. Two courses of hooks 402 each approximately 50.8 mm in width and running the length of the stretch board 400 are positioned symmetrically about the centerline 404 of the stretch board 400 such that there is a 12-mm gap (shown as gap “G”) between the courses of hooks, thereby exposing a gap of bare acrylic or polycarbonate sheet, with no hooks attached, running the entire length of the stretch board 400. Two courses of hooks 406 at least 13 mm wide and running the length of the sheet are affixed to the back side at the widthwise edge of the sheet to facilitate holding the laminate in the stretched state across the entire front-side width of the sheet.

Sample Preparation

Five like specimens representative of a sample elastomeric laminate are cut. A specimen of elastomeric laminate is a portion cut such that in its stretched state, it is at least as wide as the stretch board 400 prepared above and can be wrapped around the widthwise edges of the stretch board 400 and be secured by the back-side hook material 406. Each specimen of elastomeric laminate may be taken from roll stock or, if roll stock is not available, excised from a finished disposable absorbent article.

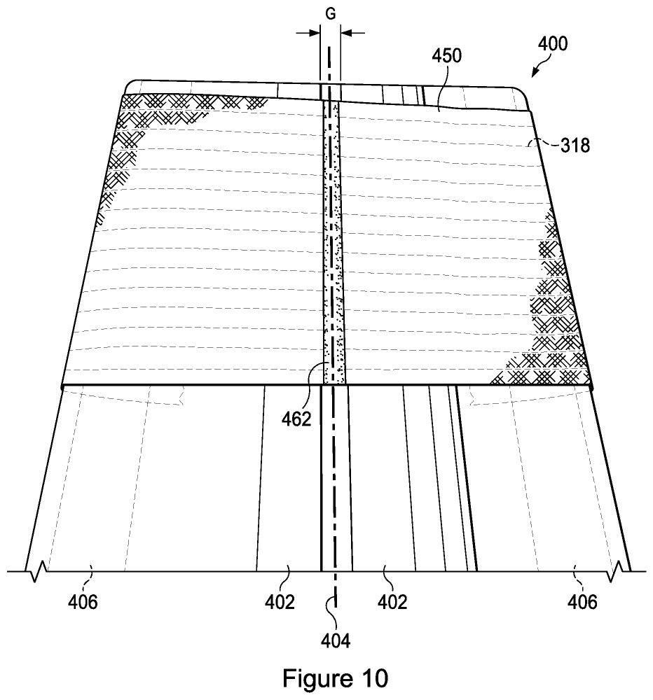

Each elastomeric laminate specimen is stretched fully and placed on a stretch board 400 such that the elastic strands 318 run the widthwise direction of the stretch board 400, perpendicular to the courses of hooks 402 running the length of the stretch board, as shown by specimen 450 in FIG. 10. The specimen 450 is held in the stretched positioned by the two courses of hook material 402 running the length of the stretch board 400. While the specimen 450 is still in the stretched state, the ends of the specimen are wrapped around the widthwise edges of the stretch board 400 and secured to the back-side hook material 406 to hold the entire laminate of the specimen 450 in the stretched state.

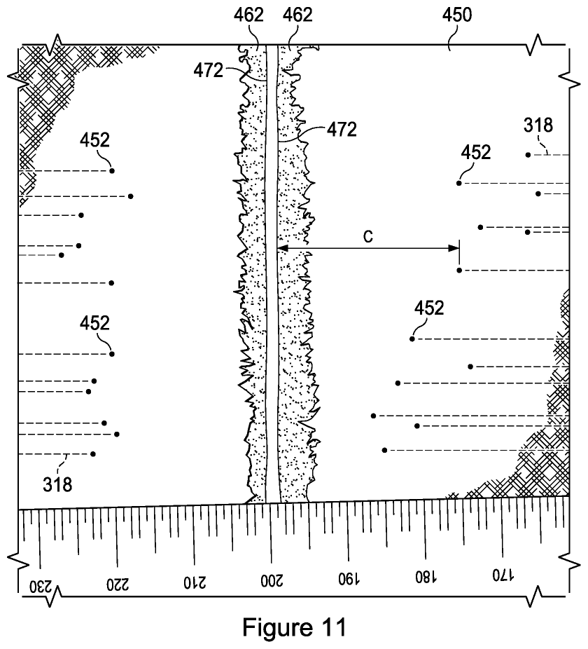

A black permanent marker is used to mark a line 462 (FIGS. 10 and 11) that is 5 mm in width direction of the specimen laminate. The black marker is applied heavily enough such that the underlying constituent elastic strands 318 of the elastomeric laminate are blackened. The line is runs in the lengthwise direction of the stretch board and is centered in the 12-mm gap between the two courses of hook material 402 position at the center of the stretch board 400 and running lengthwise. A utility knife or razor blade is then used to cut the laminate at the center of this 5-mm-wide line 462 along centerline 404. The stretch board 400 with cut specimen laminate affixed is then placed in an oven at 38° C. for 120 minutes.

Measurement and Analysis

After the stretch board 400 has been in the oven for 120 minutes, it is removed and immediately analyzed, as shown in FIG. 11. The ends of elastic strands 318 that have undergone significant creep from their starting, stretched position are evident as black dots 452 that have migrated away from the centerline 404. The widthwise distance from the cut edge 472 to each of the displaced black dots (distance “C”) is recorded to the nearest millimeter. In all, five like elastomeric laminate specimen replicates are analyzed in this way. The arithmetic mean of the displacement distances recorded among the five replicate specimens is calculated and is reported to the nearest millimeter as the “Laminate Creep.”

Static Peel Force Time Test Method (“Static Peel Force Time”)

The Static Peel Force Time Test Method is used to determine the time required for an elastomeric laminate to completely delaminate in an approximate 180° peel geometry under constant load and at fixed temperature. The peel is performed such that the crack of the peel propagates parallel to the elastic strands of the elastomeric laminate. Multiple specimens of a representative sample elastomeric laminate are taken from roll stock (if available) or one or more disposable absorbent articles and are analyzed to establish the Static Peel Force Time.

Sample Preparation

If the elastomeric laminate is available in roll stock, ten specimens measuring 27 mm in the machine direction and 25.4 in the cross direction are taken at random from the equilibrated roll stock. If an exemplary laminate is not available as roll stock, laminate specimens are excised from one or more finished disposable absorbent article(s). In this case, specimens must measure 27 mm in length parallel to the direction of elastic strands and 25.4 mm perpendicular to the direction of elastic strands.

For each specimen, the nonwoven layers of the laminate are manually peeled back 10 to 15 mm in the direction parallel to the elastic strands. (Free spray may be used very locally to enable separation of nonwoven.) For each replicate, the dimension of the remaining bonded area parallel to the direction of the elastic strands is measured to the nearest 1 mm and recorded. Regardless of whence specimens for peel analysis are sourced, each of the unbonded layers at the edge of the laminate is separately folded over a small round wooden dowel rod 2 mm in diameter and approximately 40 mm long and the wrapped dowels are secured with a 2-inch-wide bulldog clip. The clip is placed over the wrapped dowel and clamped onto the doubled layer of material such that the material does not slip or pull out of the clip.

Measurement

With clips attached, the test specimens are placed in a preconditioned incubation chamber (at 38±1° C.) for about 2 hours before testing. After 2 hours, each sample is suspended in the chamber by the clip attached to one laminate layer, and a weight is attached to the other laminate layer's clip, hanging therefrom. The hanging weight, the bulldog clip, and the dowel have a total mass of 200±2 g.

Each specimen is suspended such that the bottom of the attached weight is located high enough above the bottom of the chamber so that the entire laminate can peel apart, and the weight can freely fall to the bottom of the chamber through some remaining distance. A timer is used to measure the time between the time at which the hanging weight is attached and the time at which the bonded area of the test laminate fully delaminates. For each specimen, this time to failure is recorded to the nearest minute.

Analysis and Reporting

For each specimen, the time to failure is normalized to a 10 mm bond dimension to establish that specimen's normalized hang time, recorded for each specimen to the nearest minute.

$$Normalized hang time [ min ] = \frac{10 mm}{Bond dimension [ mm ] parallel to direction of peel} - \times time to failure [ min ]$$

The arithmetic mean of the normalized hang time values for the ten specimens is calculated and reported as the Static Peel Force Time in minutes to the nearest minute.

Average-Decitex (“Average Dtex”)

The Average-Decitex Method is used to calculate the Average Dtex on a length-weighted basis for elastic fibers present in an entire article, or in a specimen of interest extracted from an article. The decitex value is the mass in grams of a fiber present in 10,000 meters of that material in the relaxed state. The decitex value of elastic fibers or elastomeric laminates containing elastic fibers is often reported by manufacturers as part of a specification for an elastic fiber or an elastomeric laminate including elastic fibers. The Average Dtex is to be calculated from these specifications if available. Alternatively, if these specified values are not known, the decitex value of an individual elastic fiber is measured by determining the cross-sectional area of a fiber in a relaxed state via a suitable microscopy technique such as scanning electron microscopy (SEM), determining the composition of the fiber via Fourier Transform Infrared (FT-IR) spectroscopy, and then using a literature value for density of the composition to calculate the mass in grams of the fiber present in 10,000 meters of the fiber. The manufacturer-provided or experimentally measured decitex values for the individual elastic fibers removed from an entire article, or specimen extracted from an article, are used in the expression below in which the length-weighted average of decitex value among elastic fibers present is determined.

The lengths of elastic fibers present in an article or specimen extracted from an article is calculated from overall dimensions of and the elastic fiber pre-strain ratio associated with components of the article with these or the specimen, respectively, if known. Alternatively, dimensions and/or elastic fiber pre-strain ratios are not known, an absorbent article or specimen extracted from an absorbent article is disassembled and all elastic fibers are removed. This disassembly can be done, for example, with gentle heating to soften adhesives, with a cryogenic spray (e.g. Quick-Freeze, Miller-Stephenson Company, Danbury, Conn.), or with an appropriate solvent that will remove adhesive but not swell, alter, or destroy elastic fibers. The length of each elastic fiber in its relaxed state is measured and recorded in millimeters (mm) to the nearest mm

Calculation of Average Dtex

For each of the individual elastic fibers fᵢ of relaxed length Lᵢ and fiber decitex value dᵢ (obtained either from the manufacturer's specifications or measured experimentally) present in an absorbent article, or specimen extracted from an absorbent article, the Average Dtex for that absorbent article or specimen extracted from an absorbent article is defined as:

$$Average Dtex = \frac{{\sum }_{i = 1}^{n} ( {L}_{i} \times {d}_{i} )}{{\sum }_{i = 1}^{n} {L}_{i}}$$

where n is the total number of elastic fibers present in an absorbent article or specimen extracted from an absorbent article. The Average Dtex is reported to the nearest integer value of decitex (grams per 10 000 m).

If the decitex value of any individual fiber is not known from specifications, it is experimentally determined as described below, and the resulting fiber decitex value(s) are used in the above equation to determine Average Dtex.

Experimental Determination of Decitex Value for a Fiber

For each of the elastic fibers removed from an absorbent article or specimen extracted from an absorbent article according to the procedure described above, the length of each elastic fiber Lk in its relaxed state is measured and recorded in millimeters (mm) to the nearest mm Each elastic fiber is analyzed via FT-IR spectroscopy to determine its composition, and its density ρₖ is determined from available literature values. Finally, each fiber is analyzed via SEM. The fiber is cut in three approximately equal locations perpendicularly along its length with a sharp blade to create a clean cross-section for SEM analysis. Three fiber segments with these cross-sections exposed are mounted on an SEM sample holder in a relaxed state, sputter coated with gold, introduced into an SEM for analysis, and imaged at a resolution sufficient to clearly elucidate fiber cross-sections. Fiber cross-sections are oriented as perpendicular as possible to the detector to minimize any oblique distortion in the measured cross-sections. Fiber cross-sections may vary in shape, and some fibers may consist of a plurality of individual filaments. Regardless, the area of each of the three fiber cross-sections is determined (for example, using diameters for round fibers, major and minor axes for elliptical fibers, and image analysis for more complicated shapes), and the average of the three areas aₖ for the elastic fiber, in units of micrometers squared (μm²), is recorded to the nearest 0.1 μm². The decitex dₖ of the kth elastic fiber measured is calculated by:

dₖ=10,000 m×aₖ×ρₖ×10⁻⁶

where dₖ is in units of grams (per calculated 10,000 meter length), aₖ is in units of μm², and ρₖ is in units of grams per cubic centimeter (g/cm³). For any elastic fiber analyzed, the experimentally determined Lk and dₖ values are subsequently used in the expression above for Average Dtex.

Average Strand Spacing

Using a ruler calibrated against a certified NIST ruler and accurate to 0.5 mm, measure the distance between the two distal strands within a section to the nearest 0.5 mm, and then divide by the number of strands in that section −1

Average Strand Spacing=d/(n−1) where n>1

report to the nearest 0.1 mm

What is claimed is:

1. A disposable absorbent article in the form of a diaper or absorbent pant, comprising a liquid permeable topsheet, a liquid impermeable backsheet, and an absorbent core disposed between the topsheet and the backsheet, and comprising:
an elastomeric laminate, the elastomeric laminate comprising a plurality of elastic strands spaced from each other and joined with a nonwoven web material by an adhesive;
wherein the elastic strands comprise a strand polymer, wherein the strand polymer has a solubility parameter within the range of about 18 MPa^{1/2} to about 18.5 MPa^{1/2};
wherein the adhesive comprises an adhesive polymer, wherein the adhesive polymer has a solubility parameter within the range of about 16 MPa^{1/2} to about 17.5 MPa^{1/2}; and
wherein the elastic strands are sourced from a wound supply of elastic strands, the wound supply of elastic strands comprising a control layer having a solubility parameter within the range of about 15.5 MPa^{1/2} to about 16.5 MPa^{1/2} and a number average molecular weight within the range of about 0.6 kg/mol to about 1.5 kg/mol.

2. The disposable absorbent article of claim 1, wherein the number average molecular weight of the control layer is lower than the molecular weight of each of the strand polymer and the adhesive polymer.

3. The disposable absorbent article of claim 2, wherein the χN value between the control layer and the strand polymer is greater than 2 and the χN value between the control layer and the adhesive polymer is less than 2, wherein N is a degree of polymerization of the control layer and χ is a Flory-Huggins interaction parameter.

4. The disposable absorbent article of claim 1, wherein the strand polymer has a solubility parameter of about 18.3 MPa^{1/2}.

5. The disposable absorbent article of claim 1, wherein when the elastomeric laminate is subjected to the Laminate Creep Test, the elastomeric laminate has a Laminate Creep of 5 millimeters or less.

6. The disposable absorbent article of claim 5, wherein the elastomeric laminate has a Static Peel Force Time of greater than 700 min/10 mm.

7. The disposable absorbent article of claim 1, wherein the adhesive at 38° C. has a plateau modulus of elasticity of about 0.1 MPa at 38° C. and 1 Hz.

8. The disposable absorbent article of claim 1, the tensile modulus of the elastic strand at room temperature is within the range of about 5 MPa to about 15 MPa and wherein the plateau modulus of the adhesive at 38° C. is 0.1 MPa or less at 38° C. and 1 hz.

9. The disposable absorbent article of claim 8, the tensile modulus of the elastic strand at room temperature is about 10 MPa.

10. The disposable absorbent article of claim 1, wherein the control layer is a mineral oil.

11. The disposable absorbent article of claim 10, wherein the mineral oil is a paraffinic mineral oil.

12. The disposable absorbent article of claim 1, wherein the control layer comprises any of white mineral oil, polyisoprene, and polybutadiene.

13. The disposable absorbent article of claim 1, wherein the control layer is a synthetic oil.

14. The disposable absorbent article of claim 1, wherein the control layer comprises a soap.

15. The disposable absorbent article of claim 14, wherein the soap is magnesium stearate.

16. The disposable absorbent article of claim 14, wherein the control layer comprises about 1% to 5% by weight of the soap.

17. The disposable absorbent article of claim 1, wherein the elastomeric laminate has a Force Relaxation Over Time of from about 5% to about 30%.

18. The disposable absorbent article of claim 1, wherein the wound supply of elastic strands is a beam of beamed elastics.

19. The disposable absorbent article of claim 1, wherein the control layer surrounds an exterior surface of the elastic strands.

20. A disposable absorbent article in the form of a diaper or absorbent pant, comprising:
an elastomeric laminate, the elastomeric laminate comprising a plurality of laterally-spaced elastic strands joined with at least a first layer of nonwoven web material by an adhesive,
wherein the elastic strands comprise a first block copolymer of the spandex-type, wherein the block copolymer comprises a rubber block and a rigid block, wherein the rubber block is selected from a group consisting of polyethers, polyesters, and combinations thereof;
wherein the adhesive comprises an adhesive polymer, wherein the adhesive polymer comprises a second block copolymer of the styrenic type, wherein the second block copolymer comprises a rubber block, wherein the rubber block is selected from a group consisting of polyisoprene, polybutadiene, polyisoprene-co-butadiene, and hydrogenated variants thereof; and
a control layer at least partially dispersed from the elastic strand to the adhesive.

21. The disposable absorbent article of claim 20, wherein the plurality of laterally-spaced elastic strands are sourced from a beam of elastic strands.

22. A disposable absorbent article in the form of a diaper or absorbent pant, comprising:
an elastomeric laminate, the elastomeric laminate comprising a plurality of laterally-spaced elastic strands joined with at least a first layer of nonwoven web material by an adhesive,
wherein the elastic strands comprise a first block copolymer of the spandex-type, wherein the block copolymer comprises a rubber block and a rigid block, wherein the rubber block is selected from a group consisting of polyethers, polyesters, and combinations thereof;
wherein the adhesive comprises an adhesive polymer, wherein the adhesive polymer comprises a second block copolymer of the styrenic type, wherein the second block copolymer comprises a rubber block, wherein the rubber block is selected from a group consisting of polyisoprene, polybutadiene, polyisoprene-co-butadiene, and hydrogenated variants thereof; and
wherein the elastomeric laminate comprises a soap.

23. The disposable absorbent article of claim 22, wherein the soap is disposed adjacent to the elastic strands.

24. The disposable absorbent article of claim 22, wherein the soap is disposed adjacent to the adhesive.

25. A process for making an elastomeric laminate, comprising:
unwinding elastomeric strands coated with a control layer, the control layer comprising a mineral oil;
bonding the elastomeric strands between first and second substrate layers to form an elastomeric laminate;
wherein the elastomeric strands have an Average Strand Spacing from about 0.25 mm to about 4 mm; and
wherein the Average Dtex of the elastomeric strands is from about 10 to about 500.

26. The process of claim 25, wherein unwinding elastomeric strands coated with a control layer comprises unwinding the elastomeric strands from a wound supply of elastomeric strands.

27. The process of claim 26, wherein the wound supply of elastomeric strands is a beam of elastomeric strands.

28. The process of claim 25, wherein the first and second substrate layers are first and second discrete nonwovens.

29. The process of claim 25, wherein the first and second substrate layers are formed from a continuous nonwoven folded over.

30. The process of claim 25, wherein the Average Strand Spacing is from about 0.5 to about 3 mm.

31. The process of claim 25, wherein the control layer comprises a soap.

32. The process of claim 25, wherein the control layer comprises a number average molecular weight within the range of about 0.6 kg/mol to about 1.5 kg/mol.

33. The process of claim 25, further comprising:
forming at least a portion of a disposable absorbent article component using the elastomeric laminate.

34. The process of claim 33, wherein the disposable article component is selected from the group consisting of a belt, an ear, a side panel, a cuff, a waistband, a backsheet, and a topsheet.

35. A method for assembling an elastomeric laminate, the method comprising:
providing a first substrate and a second substrate;
advancing elastic strands in a machine direction, wherein the elastic strands are separated from each other in a cross direction;
applying adhesive to at least one of the elastic strands, the first substrate, and the second substrate;
combining the elastic strands with a first substrate and a second substrate to form an elastomeric laminate; and
dispersing a control layer from the elastic strands to the adhesive, wherein the control layer comprises a mineral oil.

36. The method of claim 35, further comprising:
unwinding the elastic strands from a beam.

37. The method of claim 35, wherein the elastic strands are separated from each other in the cross direction by an Average Strand Spacing from about 0.25 mm to about 4 mm.

38. The method of claim 35, wherein the Average Strand Spacing is from about 0.5 mm to about 3 mm.

39. The method of claim 35, wherein the Average Dtex of the elastic strands is from about 10 to about 500.

40. The method of claim 35, wherein the first substrate and the second substrate are discrete nonwovens.

41. The method of claim 35, further comprising:
folding a continuous nonwoven to define the first substrate and the second substrate.

42. The method of claim 35, wherein the control layer comprises a soap.

43. The method of claim 35, wherein the control layer comprises a number average molecular weight within the range of about 0.6 kg/mol to about 1.5 kg/mol.

44. The method of claim 35, further comprising:
forming at least a portion of a disposable absorbent article component using the elastomeric laminate.

45. The method of claim 44, wherein the disposable article component is selected from the group consisting of a belt, an ear, a side panel, a cuff, a waistband, a backsheet, and a topsheet.

46. The method of claim 35, wherein the elastic strands comprise a strand polymer, wherein the strand polymer has a solubility parameter within the range of about 18 MPa^{1/2} to about 18.5 Mpa^{1/2}.

47. The method of claim 35, the adhesive comprises an adhesive polymer, wherein the adhesive polymer has a solubility parameter within the range of about 16 MPa^{1/2} to about 17.5 MPa^{1/2}.
